(19) United States
(12) Patent Application Publication
(10) Pub. No.: US 2018/0161364 A1
(43) Pub. Date: Jun. 14, 2018

(54) CATIONIC COMPOUNDS AND THEIR USE AS ANTIMYCOTIC AND ANTIMICROBIAL AGENTS

(71) Applicant: CHIROBLOCK GMBH, Wolfen (DE)

(72) Inventors: Stephan Reichl, Gifhorn (DE); Dörte Von Deylen, Braunschweig (DE); Christina Dreher, Dresden (DE); Frank Leßmann, Leipzig (DE); Oliver Seidelmann, Bitterfeld (DE)

(21) Appl. No.: 15/580,252

(22) PCT Filed: Jul. 19, 2016
(86) PCT No.: PCT/EP2016/067101

§ 371 Date: Dec. 6, 2017

(30) Foreign Application Priority Data
Aug. 25, 2015 (EP) 15182310.1

(51) Int. Cl. A61K 31/787; A61K 45/06; A01N 33/12; C08G 73/02

(52) U.S. Cl. CPC A61K 31/787; A61K 45/06; A61L 12/145; C08G 73/0226; A01N 33/12

(57) ABSTRACT

The invention relates to cationic compounds and their use as antimicrobial and antimycotic agents, in particular as disinfectants and preservatives in ophthalmic preparations.

The invention relates to cationic compounds and their use as antimycotic and antimicrobial agents, in particular as disinfectants and preservatives in ophthalmic preparations.

Cosmetics and drugs for multiple use are in general subject to microbial deterioration. In order to ensure the microbial quality of such products during storage and use preservatives are added to the products. The regulatory requirements for marketing authorization and use of preservatives become more and more stringent.

At least a substantial number of the approved preservatives show significant side-effects. Due to the sensitivity of the eye this is especially critical in the ophthalmic field.

One preservative that is mainly used in ophthalmics is benzalkonium chloride (BAC). It exhibits a broad activity spectrum (also against fungi and yeasts) in a wide pH range with a low allergenic potential. However, BAC interferes with the tear film of the eye what may result in the dry eye symptom. In addition, BAC may have cytotoxic effects.

Alternative preservatives, such as Polyquad, a polycationic polymer, which is first described in U.S. Pat. No. 3,931,319 and sodium chlorite are less toxic than BAC but have a diminished activity spectrum, in particular against fungi including yeasts. Therefore, a combination with boric acid or boric acid salts is used in commercial products at the expense of compatibility (non-toxicity).

Also other polycationic compounds or polymers are used as antimicrobial agents. For example, EP 676 437, WO 02/080939 and WO 2004/046109 (US 2006/002887) disclose piperidinium ionenes and pyridinium ionenes for the treatment of microbial infections and for disinfection of medical devices, implants and the like. DE 19646726, EP 1 050 304, U.S. Pat. No. 5,512,597, WO 90/09405, WO 91/09523 and WO 2013/138820 describe polymeric, quarternary ammonium salts which are useful as preservatives or disinfectants for ophthalmic devices, such as contact lenses. According to DE 2930865, polymers having quaternary ammonium groups are used as disinfectants. WO 2013/064798 describes the use of polycationic polymers in wood preservative formulations. Z. Naturforsch. 39b, 74-78 (1984) and 43b, 778-784 (1988) disclose the preparation of 1,1″-alkanediyl-bis-3,3′- and -4,4′-bipyridine salts and their physico-chemical properties.

The agents known from the prior art have the disadvantage that their activity spectrum and/or compatibility is not fully satisfying. The problem underlying the invention is therefore to provide further agents that exhibit a broad activity spectrum against bacteria and fungi and are of acceptable compatibility so that they can also be used in ophthalmic formulations.

### SUMMARY OF THE INVENTION

This problem is solved by the cationic compounds of formula (I) as defined in the following embodiments:
1. Cationic compounds of formula (I)

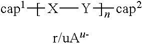

wherein
a) X is

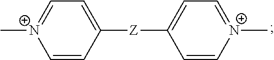

Y is C₄-C₆ alkenylene;
Z is C₁-C₆ alkylene;
cap¹ is

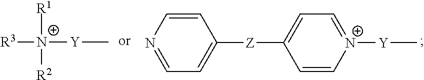

cap² is

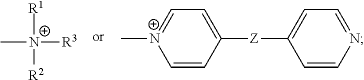

R¹, R² and R³ are independently of each other C₁-C₆ alkyl or C₁-C₆ hydroxyalkyl or one of R¹, R² and R³ is C₈-C₁₈ alkyl and the other two are independently of each other C₁-C₆ alkyl or C₁-C₆ hydroxyalkyl; and
n is 1 to 250;
b) X is

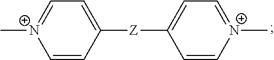

Y is C₂-C₆ alkylene; or
X—Y is a bond
cap¹ is

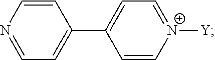

cap² is

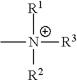

or a halogen atom;
R¹ and R² are independently of each other C₁-C₆ alkyl;
R³ is C₈-C₁₈ alkyl; and
n is 0 to 20; and
c) X is

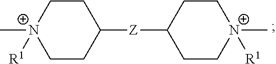

Y is C₂-C₆ alkylene;
Z is C₁-C₆ alkylene which is different from Y;
cap¹ is

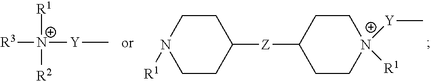

cap² is

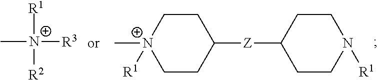

R¹, R² and R³ are independently of each other C₁-C₆ alkyl; and
n is 30 to 150;
d) X is

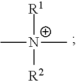

Y is C₂-C₆ alkylene;
cap¹ is

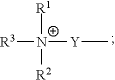

cap² is

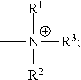

R¹ and R² are independently of each other C₁-C₆ alkyl;
R³ is C₉-C₁₈ alkyl; and
n is 10 to 500;
A is an anion;
u is the valency of the anion; and
r is the number of positive charges in the compound.
2. A cationic compound of embodiment 1, corresponding to the compounds of group (d) having the formula (Id):

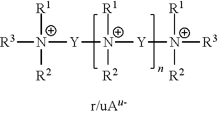

wherein n, r, u, A, Y, R¹, R² and R³ are as defined in embodiment 1.
3. A cationic compound of embodiment 2, wherein R¹ and R² are methyl.
4. A cationic compound of embodiment 2 or 3, wherein R³ is C₁₂-C₁₆ alkyl.
5. A cationic compound of any one of embodiments 2 to 4, wherein Y is C₃-C₄ alkylene.
6. A cationic compound of any one of embodiments 2 to 5, wherein n is 100 to 400, in particular 90 to 140.
7. A cationic compound of embodiment 1, corresponding to the compounds of group (a) having the formula (Ia):

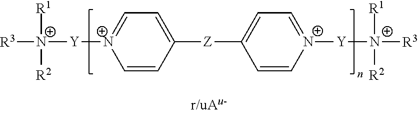

wherein n, r, u, A, Y, Z, R¹, R² and R³ are as defined in embodiment 1.
8. A cationic compound of embodiment 7, wherein R¹, R² and R³ are methyl or ethyl.
9. A cationic compound of embodiment 7 or 8, wherein Y is C₄-alkenylene.
10. A cationic compound of any one of embodiments 7 to 9, wherein Z is C₂-C₄ alkylene.
11. A cationic compound of any one of embodiments 7 to 10, wherein n is 170 to 220.
12. A cationic compound of embodiment 1 corresponding to the compounds of group (b) having the formula (Ib):

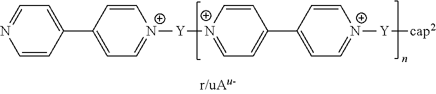

wherein n, r, u, A, Y and cap² are as defined in embodiment 1.
13. A cationic compound of embodiment 12, wherein cap² is

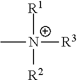

and R¹ and R² are methyl and R³ is as defined in embodiment 1.
14. A cationic compound of embodiment 13, wherein R³ is C₁₀-C₁₆ alkyl, in particular C₁₀-C₁₂ alkyl and preferably C₁₀ alkyl or Cᵢᵢ alkyl.
15. A cationic compound of any one of embodiments 12 to 14, wherein Y is C₂-C₄ alkylene.
16. A cationic compound of any one of embodiments 12 to 15, wherein n is 0 to 2, in particular 0.
17. A cationic compound of embodiment 12, wherein n is 0 and cap² is a halogen atom, in particular a chlorine or bromine atom.
18. A cationic compound of embodiment 1 corresponding to the compounds of group (c) having the formula (Ic):

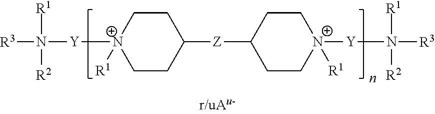

wherein n, r, u, A, Y, R¹, R² and R³ are as defined in embodiment 1.
19. A cationic compound of embodiment 18, wherein R¹, R² and R³ are methyl.
20. A cationic compound of embodiments 18 or 19, wherein Z is C₁-C₃ alkylene.
21. A cationic compound of any one of embodiments 18 to 20, wherein Y is C₄-C₆ alkylene.
22. A cationic compound of any one of embodiments 18 to 21, wherein n is 90 to 140.
23. A composition comprising a cationic compound of any one of embodiments 1 to 22.
24. A composition of embodiment 23 in the form of a disinfectant formulation for medical devices, in particular contact lenses.
25. A composition of embodiment 23 in the form of a preservative formulation for liquid, semi-solid or solid drugs, ophthalmic formulations or cosmetics.
26. An ophthalmic composition comprising a cationic compound of any one of embodiments 1 to 22.
27. A composition of any one of embodiments 23 to 26 which comprises 0.01 to 1.0 wt.-% of a cationic compound of any one of embodiments 1 to 22.
28. A composition of any one of embodiments 23 to 27 which comprises an additional disinfectant or preservative.
29. The use of a cationic compound of any one of embodiments 1 to 22 or of a composition of any one of embodiments 23 to 28 as a disinfectant or preservative.
30. A method of disinfecting or preserving liquid, semi-solid or solid drug formulations, medical devices, in particular contact lenses, or cosmetics which comprises adding a cationic compound of any one of embodiments 1 to 22 or of a composition of any one of embodiments 23 to 28 to said drug formulations or cosmetics or contacting said medical devices, in particular contact lenses, with a cationic compound of any one of embodiments 1 to 22 or of a composition of any one of embodiments 23 to 28.
31. The use of a cationic compound of any one of embodiments 1 to 22 or of a composition of any one of embodiments 23 to 28 as an antimycotic agent.
32. A cationic compound of any one of embodiments 1 to 22 or of a composition of any one of embodiments 23 to 28 for use as an antimycotic agent.
33. The use of a cationic compound of any one of embodiments 1 to 22 or of a composition of any one of embodiments 23 to 28 as an antibacterial agent.
34. A cationic compound of any one of embodiments 1 to 22 or of a composition of any one of embodiments 23 to 28 for use as an antibacterial agent.

### BRIEF DESCRIPTION OF THE DRAWINGS

FIG. 1 shows the course of the TEER value for the compound of example 2

FIG. 2 shows the course of the TEER value for the compound of example 3

FIG. 3 shows the course of the TEER value for the compound of comparative example 1

FIG. 4 shows the course of the TEER value for the compound of comparative example 2

FIG. 5 shows the course of the TEER value for the compound of example 5

FIG. 6 shows the course of the TEER value for the compound of example 4

FIG. 7 shows the course of the TEER value for the compound of example 1.2

### DETAILED DESCRIPTION OF THE INVENTION

The term “alkyl” as used herein means a straight or branched alkyl group having a number of carbon atoms as indicated. Examples for “alkyl” are methyl, ethyl, n-propyl, isopropyl, n-butyl, isobutyl, t-butyl, n-pentyl or n-hexyl. Further examples are n-nonyl, isononyl, n-decyl, n-dodecyl, n-tridecyl, isotridecyl, n-tetradecyl, n-hexadecyl and n-octadecyl.

The term “alkylene” as used herein means a saturated, straight or branched hydrocarbon group derived by the removal of two hydrogen atoms from the same or two different carbon atoms of a parent alkane. The number of carbon atoms is as indicated. Examples for “alkylene” are methylene (—CH₂—), 1,2-ethylene (—CH₂CH₂—), 1,1-propylene (—CH(CH₂CH₃)—), 1,2-propylene (—CH₂CH(CH₃)—), 1,3-propylene (—CH₂CH₂CH₂—), 1,4-butylene (—CH₂CH₂CH₂CH₂—), isobutylene (—CH(CH₃)CH₂CH₂—), 2-methylpropylene (—CH₂CH(CH₃)CH₂—), n-pentylene (—CH₂CH₂CH₂CH₂CH₂—) or n-hexylene (—CH₂CH₂CH₂CH₂CH₂CH₂—).

The term “alkenylene” as used herein means an unsaturated, straight or branched hydrocarbon group derived by the removal of two hydrogen atoms from the same or two different carbon atoms of a parent alkene. An alkenylene group as used herein has four to six carbon atoms and one double bond. The double bond is not terminal and the term includes E and Z isomers. Examples for “alkenylene” are 2-butenylene (—CH₂CH═CHCH₂—), 2-methyl-2-butenylene (—CH₂C(CH₃)═CHCH₂—), 2-pentenylene (—CH₂CH═CHCH₂CH₂—), 2-methyl-2-pentenylene (—CH₂C(CH₃)═CHCH₂CH₂—) or 2-methyl-3-pentenylene (—CH₂CH(CH₃)CH═CHCH₂—).

Examples for the term “anion” are inorganic anions such as Cl⁻, Br⁻, I⁻, SO₄²⁻, HSO₄⁻, CO₃²⁻, HCO₃⁻, PO₄³⁻, HPO₄²⁻, or H₂PO₄⁻or organic ions derived from carboxylic or sulfonic acids such as acetate, sorbate or methylsulfonate. Br⁻ is preferred.

The cationic compounds of the invention can be prepared as follows:

### Compounds of Group (a):

A α,ω-alkylenedipyridine compound is reacted under heating with a α,ω-dihalogeno-C₄-C₆-alkene in an aprotic or protic polar solvent, such as ketones like acetone; acetonitrile; ethylacetate; C₁-C₄ alkanols like methanol, ethanol, n-propanol, isopropanol, n-butanol, isobutanol or t-butanol; dimethylformamide or water or mixtures thereof, and in the presence of a tri-C₁-C₆ alkylamine. The α,ω-dihalogeno-C₄-C₆-alkene is preferably a α,ω-dichloro or α,ω-dibromo-C₄-C₆-alkene.

### Compounds of Group (b):

4,4′-bipyridine is reacted under heating with an α,ω-dihalogeno-C₁-C₆-alkane in an aprotic or protic polar solvent, as given under (a) above, and in the presence of a di-C₁-C₆-alkyl-C₈-C₁₈-alkylamine. As dihalogeno-C₁-C₆-alkane a dichloro or dibromo-C₁-C₆-alkane is used.

### Compounds of Group (c):

A α,ω-C₂-C₆-alkylene-N,N′-di-C₁-C₆-alkyldipiperidine is reacted under heating with an α,ω-dihalogeno-C₁-C₆-alkane in an aprotic or protic polar solvent, as given under (a) above, and in the presence of a tri-C₁-C₆-alkylamine. As dihalogeno-C₁-C₆-alkane a dichloro or dibromo-C₁-C₆-alkane is used.

### Compounds of Group (d):

A N,N,N′,N′-tetra-C₁-C₆-alkyl-α,ω-C₁-C₆-alkane diamine is reacted under heating with an α,ω-dihalogeno-C₁-C₆-alkane in an aprotic or protic polar solvent, as given under (a) above, and in the presence of a di-C₁-C₆-alkyl-C₉-C₁₈-alkylamine. As dihalogenoalkane a dichloro or dibromo-C₁-C₆-alkane is used.

The compounds of the invention are cationic, including polycationic, compounds having antimicrobial and antimycotic activity against bacteria and, in particular, fungi including yeasts. It was most surprising that the compounds of the invention have also activity against aspergillus brasiliensis (aspergillus niger). Furthermore, the compounds of the invention are highly compatible with sensitive tissues and non-irritating. The compounds of the invention are therefore useful as preservatives of formulations (compositions) that are subject to deterioration by bacteria, yeasts or fungi. Such formulations are in particular those for multiple use such as liquid drug formulations such as aqueous or non-aqueous solutions or suspensions, semi-solid drug formulations for topical treatment such as creams or ointments, solutions for disinfecting medical devices, or cosmetics. Preferably, the compounds of the invention are used in ophthalmic formulations such as solutions that are directly applied to or in contact with ocular tissues or solutions for treating ophthalmic devices such as contact lenses or other ophthalmic devices. Such solutions include, for example, eye drops, eyewash solutions, and solutions for contact lens care such as cleaning solutions or storing solutions, and solutions for disinfecting other ophthalmic devices.

The compounds of the invention are further useful as active compounds in other medicinal formulations such as ophthalmic solutions for treating the eye, nose drops, or antimycotic compositions or in disinfecting formulations.

The compounds of the invention are also useful as preservative for wood, leather or food products as well as for cosmetics such as shampoos, hand and body lotions, moisturizing and cleansing emulsions, antiperspirants, deodorants, and the like.

The present compositions or formulations comprising the compounds of the invention can be formulated, for example, as disinfecting compositions, cleaning compositions, wetting compositions, conditioning compositions, soaking compositions and, in particular, ophthalmic compositions. Also, the present compositions can be formulated to be useful in performing two or more contact lens caring operations such as a disinfecting/cleaning composition, or a cleaning/conditioning composition or even an all purpose lens care composition.

The compounds of the present invention are usually contained in a formulation or composition of the invention at a concentration ranging from 0.0001 to 1.0 w/v %, preferably from 0.001 to 0.1 w/v %, weight per volume of the formulation or composition. For example, disinfectants for ophthalmic devices are formulated using the compounds usually at a concentration ranging from 0.0001 to 1.0 w/v %, preferably from 0.001 to 0.1 w/v %. Eye drops, eyewash solutions, or cleaning solutions, storing solutions, or cleaning-storing solutions used for contact lens care are formulated using the compounds usually at a concentration ranging from 0.0001 to 0.01 w/v %, preferably from 0.0005 to 0.005 w/v %.

There is no restriction on the pH of the compositions or formulations of the present invention as long as the pH is within the physiologically, in particular ophthalmologically acceptable range; the pH value usually ranges from about 5.0 to 9.0, preferably from 5.5 to 8.5. The osmotic pressure ratio of ophthalmic formulations (the ratio of osmotic pressure of the ophthalmic solution to the osmotic pressure of physiological saline) is usually adjusted to about 0.5 to 5.0, preferably to about 0.8 to 2.0.

The ophthalmic solutions of the present invention can be formulated with various ingredients besides the compounds of the invention. There are no restrictions on the ingredients contained in the solutions. For example, the formulations may be formulated with a variety of additives such as buffering agents, isotonizing agents, solubilizers, stabilizers, viscoelastic agents, chelating agents, and pH-adjusting agents as well as active ingredients such as agents for removing congestion, anti-inflammatory agents, astringents, antihistaminic agents, anti-microbial agents, glaucomatosa, steroids, vitamins, amino acids, inorganic salts, and saccharides. The buffering agents include, for example, borate buffer, phosphate buffer, carbonate buffer, acetate buffer, citrate buffer, ε-aminocapronic acid, glutamic acid and salts thereof, and aspartic acid and salts thereof. The isotonizing agents include, for example, sodium chloride, potassium chloride, calcium chloride, glycerol, glucose, mannitol, aminoethyl sulfonic acid, aspartic acid, potassium aspartate, sodium aspartate, and magnesium potassium aspartate. In particular, the preferable isotonizing agents are aspartic acid and/or salts thereof; the salts preferred are sodium aspartate, potassium aspartate, and magnesium aspartate. The ophthalmic solutions are formulated with a solution of aspartic acid and/or salts thereof that is isotonic with 0.5 to 2.0% sodium chloride solution.

The present compositions may include other, e.g., complementary and/or potentiating, antimicrobial agents. Examples of such other antimicrobial agents include thimerosal, sorbic acid, 1.5-pentanedial, alkyl triethanolamines, boric acid, other polycationic compounds such as benzalconium chloride, physiologically acceptable salts of any of the above, 3-chloroallyl-3,5,7-triaza-1-azoniaadamantine chloride, phenylmercuric salts and mixtures thereof.

Thus, the present compositions can be formulated, for example, as disinfecting compositions, cleaning compositions, wetting compositions, conditioning compositions, soaking compositions. Also, the present compositions can be formulated to be useful in performing two or more contact lens caring operations such as a disinfecting/cleaning composition, or a cleaning/conditioning composition or even an all purpose lens care composition.

The following examples illustrate the invention without limiting it.

The molar mass M_{w} of polymers where given was determined by GPC (gel permeation chromatography) as follows. Suitable columns are those charged with polyacrylate/methacrylate beads that are surface-modified by NH-functionalization such as columns of the Novema Max series which are commercially available from PSS Polymer Standards Service GmbH, Mainz, Germany. Two columns (Novema Max, 10 μm and 30 Å or 1000 Å) were used in series and eluted as described below.

### Measuring Conditions:

Solvent: buffer A (0.1% trifluoro acetic acid, 0.05% NaN₃ in water) and buffer B (acetonitrile) in a ratio of 90:10 v/v.
Flow: 1 ml/min at about 80 bar.

### Detection: RID

### Concentration: 5 g/I

Injection volume: 100 μl
Calibration: pullulan available from PSS (10 standards with molar peaks from 342 Da to 708000 Da).

### Example 1

### Synthesis of Polycationic Compound (3a) and (3b)

### 1.1 Polycationic Compound (3a)

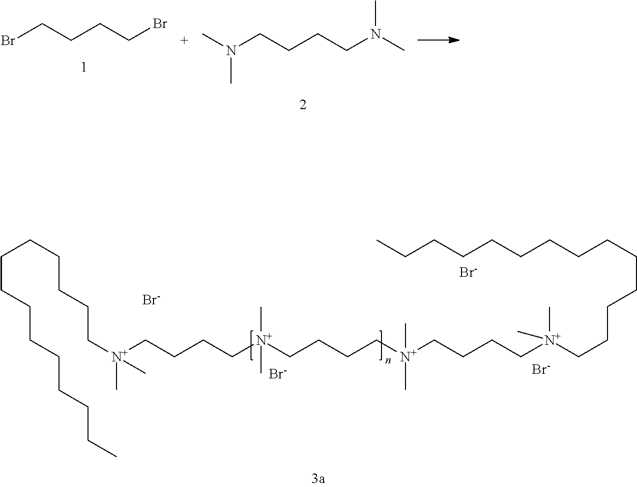

A 100 ml flask equipped with reflux condenser, magnetic stirrer and adsorber tube was charged with 1.26 ml N,N,N′N-tetramethyl-1,4-butane diamine (1.0 g, 6.9 mmol) in 40 ml acetone (HPLC grade). Then, 1.2 ml N,N-dimethyltetradecylamine (0.96 g, 4.0 mmol, 0.57 equiv.) and 0.91 ml 1,4-dibromobutane (1.65 g, 7.6 mmol, 1.1 equiv.) were added successively. The reaction mixture was stirred under reflux for 15 hours. Thereby a white precipitate was formed. The cooled suspension was filtered off rapidly, the filtrate was washed with 40 ml acetone (HPLC grade) and dried under vacuum at 50° C. for 30 minutes. Compound 3a was obtained as a white solid (2.85 g, 95% yield). ¹H-NMR (D2O, 500 MHz): δ (in ppm)=0.84-0.94 (br, alkyl end cap), 1.25-1.45 (br, alkyl end cap), 1.73-1.84 (br, alkyl end cap), 1.85-195 (br, 4H), 3.08-3.18 (br, 6H), 3.40-3.48 (br, 4H); M_{w} ˜42000 g·mol⁻¹ according to the GPC method.

### 1.2 Polycationic Compound (3b)

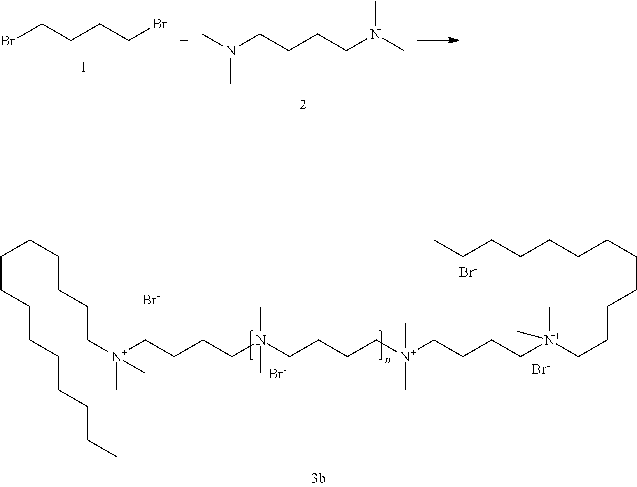

The above procedure under 1.1 was repeated using 4.0 mmol of N,N-dimethyl-dodecylamine in place of N,N-dimethyltetradecylamine to obtain compound 3b as a white solid.

### Example 2

### Synthesis of Polycationic Compound (6)

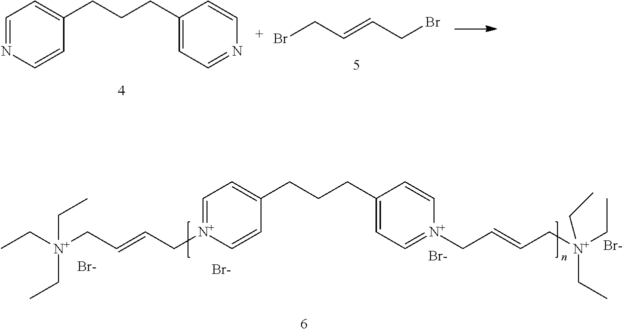

A 100 ml flask equipped with reflux condenser, magnetic stirrer and adsorber tube and charged with a solution of 2.0 g 4,4′-trimethylenedipyridine (10.1 mmol, 1.0 equiv.) in 40 ml acetone (HPLC). Then, 2.37 g trans-1,4-dibromo-2-butene (11.1 mmol, 1.1 equiv.) were added in one portion and the reaction mixture was stirred under reflux for 30 minutes. Subsequently, 0.85 ml triethylamine (0.61 g, 6.1 mmol, 0.6 equiv.) were added and the mixture was stirred under reflux for additional 5 hours. A white precipitation was formed which was filtered off rapidly, washed with 50 ml acetone (HPLC) and dried in vacuum at 50° C. for 30 minutes.

Compound 6 was obtained as a greenish powder (3.12 g, 70% yield). ¹H-NMR (D2O, 500 MHz): δ (in ppm)=1.32 (t, CH3), 2.20 (m, 2H, CH2), 3.06 (m, 4H, CH2), 3.32 (q, CH2), 5.28 (d, 4H, CH2), 6.28 (m, 2H, CH), 7.98 (d, 4H, Ar—H), 8.72 (d, 4H, Ar—H); M_{w} ˜91.000 g·mol⁻¹ according to the GPC method.

### Comparative Examples 1 and 2

In an analogous manner to example 2 the following compounds were prepared:

### Comparative Example 1

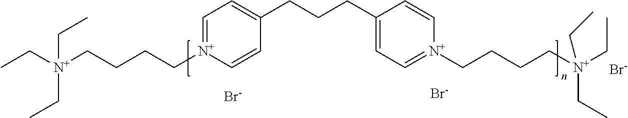

M_{w} ˜75.000 g·mol⁻¹ according to the GPC method.

### Comparative Example 2

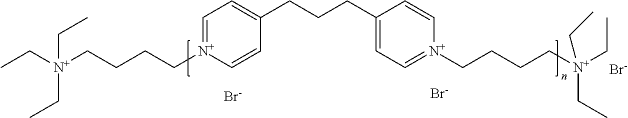

M_{w} ˜82.000 g·mol⁻¹ according to the GPC method.

### Example 3

### Synthesis of the Dimeric Compound (9)

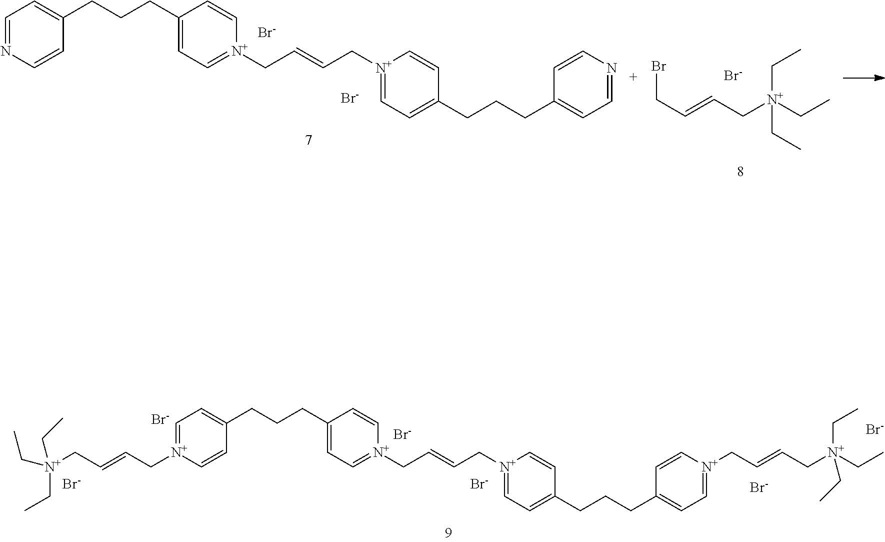

### Synthesis of the Centerpiece Substructure (7)

A 100 ml flask equipped with a magnetic stirrer was charged with 4.63 g 4,4′-trimethylenedipyridine (23.4 mmol, 10.0 equiv.) in 30 ml acetone (HPLC). Then, 0.5 g trans-1,4-dibromo-2-butene (99%, 2.3 mmol, 1.0 equiv.) were added at once and the clear solution was stirred at 20° C. overnight. Thereby the solution became turbid. The precipitate was filtered off, washed with 20 ml acetone and dried in vacuum at 50° C. The compound 7 was obtained as a green solid (1.43 g, quantitative). ¹H-NMR (D20, 500 MHz): δ (in ppm)=1.99 (m, 4H), 2.62 (t, 4H), 2.84 (t, 4H), 5.14 (d, 4H), 6.10 (m, 2H), 7.16 (d, 4H), 7.77 (dd, 4H), 8.24 (dd, 4H), 8.53 (d, 4H); ESI-MS: m/z calcd for C₃₀H₃₄N₄²⁺=450.63 for [M²⁺-2Br⁻], found: 225 (m/2).

### Synthesis of the End Piece Substructure (8)

A 100 ml flask equipped with a magnetic stirrer was charged with 3.2 g trans-1,4-dibromo-2-butene (99%, 15 mmol, 2.1 equiv.) in 40 ml acetone (HPLC). Then, 1 ml triethylamine (0.72 g, 7.1 mmol, 1.0 equiv.) were added dropwise and the clear solution was stirred at 20° C. overnight. Thereby the solution became turbid. The precipitate was filtered off, washed with 20 ml acetone and dried in vacuum at 50° C. using a rotary evaporator. The compound 8 was obtained as a white solid (2.0 g, 89% yield). ¹H-NMR (DMSO-d6, 500 MHz): δ (in ppm)=1.20 (t, 9H), 2.21 (q, 6H), 3.94 (d, 2H), 4.19 (d, 2H), 6.04 (m, 1H), 6.33 (m, 1H); ESI-MS: m/z calcd for C₁₀H₂₁BrN₃⁺=235.19 for [M⁺-Br—], found: 234, 236.

### Synthesis of Compound (9)

A flask was charged with 600 mg compound 7 (1 mmol, 1.0 equiv.) and 650 mg compound 8 (2.1 mmol, 2.1 equiv.) in 25 ml methanol (HPLC) and the mixture was stirred for 15 hours at reflux temperature. Afterwards, the solvent was removed using a rotary evaporator and the residue was washed with diethyl ether (3×50 ml). The organic extracts were discarded and the residue was freeze-dried to give compound 9 as a yellowish foam (810 mg, 65% yield). ¹H-NMR (D₂O, 500 MHz): δ (in ppm)=1.35 (t, 18H), 2.26 (m, 4H), 3.10 (m, 8H), 3.37 (q, 12H), 4.00 (d, 2H), 5.34 (d, 8H), 6.21 (m, 2H), 6.31 (q, 2H), 6.50 (m, 2H), 8.02 (t, 8H), 8.76 (t, 8H).

### Example 4

### Synthesis of Polycationic Compound (11)

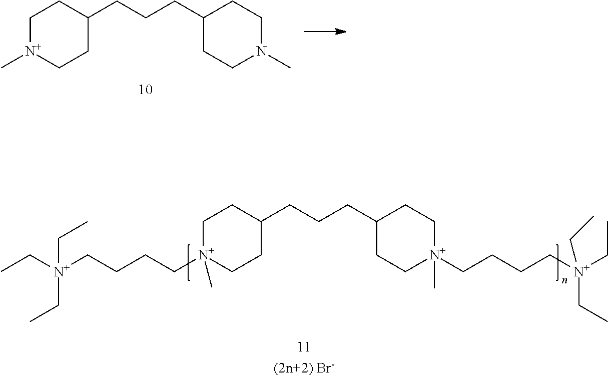

The starting material 10 was synthesized from commercially available 4,4′-trimethylene-dipiperidine according to the protocol described by A. P. Phillips in J. Amer. Chem. Soc., 1955, 76, 6396.

A 100 ml flask equipped with reflux condenser, magnetic stirrer and adsorber tube was charged with 1.0 g compound 10 (4.2 mmol, 1.0 equiv.) in 40 ml acetone (HPLC). To this solution, 0.35 ml triethylamine (0.26 g, 2.5 mmol, 0.6 equiv.) and 0.55 ml dibromobutane (1.0 g, 4.6 mmol, 1.1 equiv.) were added successively. The reaction mixture was stirred under reflux for 15 hours. Thereby a white precipitate was formed which was filtered off rapidly, washed with 50 ml acetone (HPLC) and dried under vacuum at 50° C. for 30 minutes. Compound 11 was obtained as a beige solid (1.87 g, 71% yield). ¹H-NMR (D₂O, 500 MHz): δ (in ppm)=1.25 (CH₃ (TEA)), 1.3-1.4 (br, 6H), 1.5-1.7 (br, 6H), 1.7-1.8 (br, 2H), 1.8-2.0 (br, 6H), 2.42 (CH₂ (TEA)), 3.02 (s, 3H), 3.05 (s, 3H), 3.2-3.3 (m, 4H), 3.3-3.5 (m, 8H); M_{w} ˜59.000 g·mol⁻¹ according to the GPC method).

### Example 5

### Synthesis of Compound 13

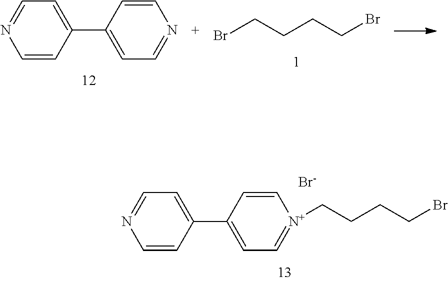 [SMILES: c1cc(-c2ccncc2)ccn1 ; BrCCCCBr ; [Br-] ; BrCCCC[n+]1ccc(-c2ccncc2)cc1]

A 100 ml flask equipped with reflux condenser, magnetic stirrer and adsorber tube was charged with 1.0 g 4,4′-bipyridine (6.4 mmol, 1.0 equiv.) in 30 ml acetone (HPLC grade). To this solution 0.77 ml 1,4-dibromobutane (1.38 g, 6.4 mmol, 1.0 equiv.) were added in one portion. The reaction mixture was stirred under reflux for 15 hours. Thereby a precipitate formed, which was filtered off rapidly, washed twice with 50 ml acetone (HPLC grade) and dried under vacuum at 50° C. for 30 minutes. Compound 13 was obtained as a yellow solid (1.87 g, 79% yield). ¹H-NMR (D₂O, 500 MHz): δ (in ppm)=1.97 (m, 2H), 2.23 (m, 2H), 3.55 (t, 2H), 4.73 (t, 2H), 7.91 (dd, 2H), 8.42 (d, 2H), 8.76 (dd, 2H), 9.00 (d, 2H); ESI-MS: m/z calcd for C₁₄H₁₆BrN₂=292.2 for [M⁺-Br⁻], found: 291; 293.

### Comparative Example 3

### Synthesis of Dimeric Compound 13a

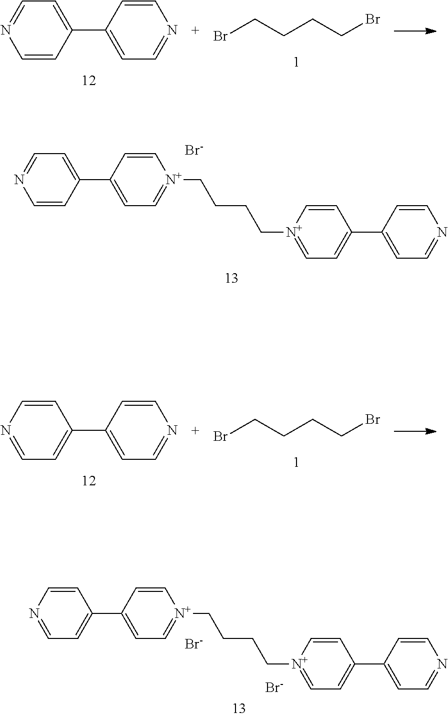

A 100 ml flask equipped with reflux condenser, magnetic stirrer and adsorber tube was charged with 1.0 g 4,4′-bipyridine (6.4 mmol, 1.0 equiv.) in 30 ml acetone (HPLC grade). To this solution, 0.68 ml 1,4-dibromobutane (1.23 g, 5.7 mmol, 0.89 equiv.) were added successively. The reaction mixture was stirred under reflux for 15 hours. Thereby a yellowish precipitate was formed which was filtered off rapidly, washed with 50 ml acetone (HPLC grade) and dried under vacuum at 50° C. for 30 minutes. The compound 13 was obtained as an orange solid (860 mg).

### Example 6.1

### Synthesis of Compound 14a

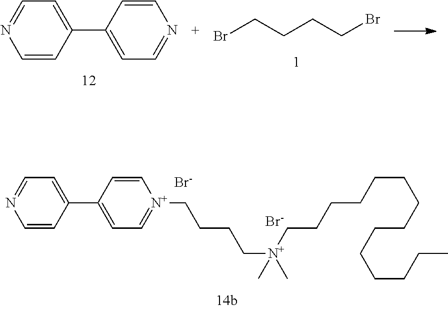 [SMILES: c1cc(-c2ccncc2)ccn1 ; BrCCCCBr ; CCCCCCCCCCCCCC[N+](C)(C)CCCC[n+]1ccc(-c2ccncc2)cc1 ; [Br-] ; [Br-]]

A 100 ml flask equipped with reflux condenser, magnetic stirrer and adsorber tube was charged with 1.0 g 4,4′-bipyridine (6.4 mmol, 1.0 equiv.) in 30 ml acetone (HPLC grade). To this solution, 0.84 ml 1,4-dibromobutane (1.52 g, 7.0 mmol, 1.1 equiv.) and 1.0 ml N,N-dimethyltetradecylamine (0.8 g, 3.3 mmol, 0.52 equiv.) were added successively. The reaction mixture was stirred under reflux for 15 hours. Thereby a yellowish precipitate formed which was filtered off rapidly, washed with 50 ml acetone (HPLC grade) and dried under vacuum at 50° C. for 30 minutes. The compound 14a was obtained as an orange solid (2.46 g, 63%). ¹H-NMR (D₂O, 500 MHz): δ (in ppm)=0.88 (t, 3H), 1.29-1.48 (m, 24H), 1.79 (m, 2H), 2.01 (m, 2H), 2.27 (s, 6H), 3.24 (t, 2H), 3.59 (t, 2H), 4.88 (t, 2H), 7.95 (m, 2H), 8.46 (dd, 2H), 8.81 (m, 2H), 9.02 (d, 2H).

### Example 6.2

### Synthesis of Compound 14b

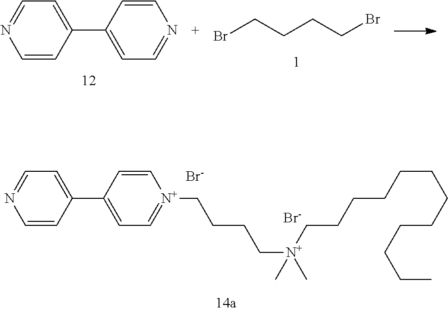 [SMILES: c1cc(-c2ccncc2)ccn1 ; BrCCCCBr ; CCCCCCCCCCCC[N+](C)(C)CCCC[n+]1ccc(-c2ccncc2)cc1 ; [Br-] ; [Br-]]

Compound 14b was prepared according to the synthesis of 14a using 1.0 g 4,4′-bipyridine (6.4 mmol, 1.0 equiv.), 0.84 ml 1,4-dibromobutane (1.52 g, 7.0 mmol, 1.1 equiv.) and 0.85 ml N,N-dimethyldodecylamine (0.67 g, 3.15 mmol, 0.49 equiv.) in 30 ml acetone (HPLC grade) and was obtained as an orange solid (1.83 g, 49%). ¹H-NMR (D₂O, 500 MHz): δ (in ppm)=0.88 (t, 3H), 1.1-1.4 (m, 20H), 1.77 (m, 2H), 2.01 (m, 2H), 2.27 (s, 6H), 3.11 (t, 2H), 3.59 (t, 2H), 4.87 (t, 2H), 7.95 (m, 2H), 8.47 (dd, 2H), 8.81 (m, 2H), 9.02 (d, 2H); ESI-MS: m/z calcd for C₂₈H₄₇N₃²⁺=425.7 for [M²⁺-2 Br⁻], found: 212 (m/2).

### Example 6.3

Compound 14c was prepared analogous to example 6.2 but using N,N-dimethyldecylamine in place of N,N-dimethyldodecylamine.

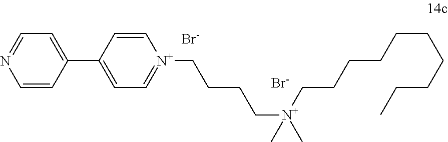 [SMILES: CCCCCCCCCC[N+](C)(C)CCCC[n+]1ccc(-c2ccncc2)cc1 ; [Br-] ; [Br-]]

The reactions described above were also carried out using 1:1 methanol:dimethylformamide (v/v) as a solvent. Under said conditions, the products were freeze-dried to obtain the compounds as oils or foams.

The minimum inhibitory concentration (MIC) of the compounds of examples 1 to 5 was determined and is given in table 1 below. The following bacteria and fungi strains and methods were used:

Aspergillus brasiliensis (2d) and (9d): MIC determined in accordance with a method of the European Committee on Antimicrobial Susceptibility Testing: EUCAST E.DEF 9.1, July 2008, “Method for the determination of broth dilution minimum inhibitory concentrations of antifungal agents for conidia forming moulds” available under www.eucast.

Candida albicans: MIC determined in accordance with the method described in EUCAST DEFINITIVE DOCUMENT EDef 7.2 “Method for the determination of broth dilution minimum inhibitory concentrations of antifungal agents for yeasts” available under www.eucast.org.

Pseudomonas aeruginosa and Escherichia coli: MIC determined in accordance with DIN EN ISO 20776-1: 2006.

TABLE 1

| | MIC (mg/l) of the compound of | | | | | | | | |
|---|---|---|---|---|---|---|---|---|---|
| Microorganism | Ex. 1.2 | Ex. 2 | Ex. 3 | Ex. 4 | Ex. 5 | Ex. 6.2 | Comp. ex. 1 | Comp. ex. 2 | Comp. ex. 3 |
| A. bras.¹⁾ (2d) | 6.3-12.5 | 1.6-6.3 | 12.5 | 3.1 | 3.1 | 6.3 | 1.6 | 1.56 | 200 |
| A. bras.¹⁾ (9d) | 6.3-12.5 | 6.3 | 12.5-25.0 | 3.1-6.3 | 3.1-50 | 6.3 | 12.5 | 3.12 | 200-400 |
| C. albicans²⁾ | 50 | <3.1 | <3.1 | <3.1 | 1.56 | <3.12 | 0.78 | <3.12 | 200 |
| P. aeruginosa³⁾ | 6.3 | 6.3 | 25 | 12.5 | 12.5 | 12.5 | 3.1 | 3.12 | >400 |
| E. coli⁴⁾ | 6.3 | 12.5 | 50 | 12.5 | 6.3 | 6.3 | 6.3 | 3.12 | 200 |

¹⁾(DSM 1988/ATCC 16404)
²⁾(DSM 1386/ATCC 10231)
³⁾(DSM 1128/ATCC 9027)
⁴⁾(DSM 1576/ATCC 8739)

As can be seen, the compounds of the invention are highly active against fungi and bacteria, even against such problematic fungi like aspergillus brasiliensis and bacteria like pseudomonas aeruginosa. The compounds of comparative examples 1 and 2 have acceptable activity but are unsuitable for practical use because they Impair the cell barrier, see the TEER values below, FIGS. 3 and 4.

The toxicity of the compounds of the invention was determined in accordance with a conventional MTT test. Human corneal epithelial cells (HCE-T-cell line) were cultivated in 5% Sasaki cell culture medium consisting of 250 ml DMEM (Dulbecco's Modified Eagle's Medium), 250 ml Ham's F12 medium, 27.17 ml fetale calf serum, 543.4 μl insulin 5 mg/ml, 1.086 ml EGF stock solution 5 μg/ml, 2.717 ml DMSO and 5.43 ml antibiotic/antimycotic solution. The cells were seeded into a 96 wells cell culture plate (18 000 cells/well) and grown in an incubator for about 24 h (37° C., 5% CO₂). The medium was removed and the test compounds dissolved in KRB (Krebs Ringer buffer) were applied. 100 μl KRB were used as positive control whereas a 0.5% Triton X solution was used as negative control.

After incubation for 10 min. under light protection the test compounds were removed and the plates were rinsed once with 100 μl phosphate buffered saline (PBS without Mg and Ca) per well. After removal of PBS 100 μl MTT reagent (0.05%) were added into each well followed by incubation for 3h at 37° C. under light protection. Thereafter, the reagent was removed and replaced by 100 μl lysis solution. The dark blue dye was dissolved from the cells by shaking. After 30 min. the plate was evaluated by UV spectroscopy at 570 nm.

The results are given in table 2 below:

TABLE 2

| Compound¹⁾ | Viability²⁾ (%) |
|---|---|
| Ex. 1.2 | 86.8 ± 7.6 |
| Ex. 2 | 93.6 ± 11.1 |
| Ex. 3 | 90.3 ± 7.0 |
| Ex. 4 | 82.3 ± 9.1 |
| Ex. 5 | 83.8 ± 12.6 |
| Ex. 6.2 | 74.9 ± 12.0 |
| Comp. ex. 1 | 83.0 ± 6.1 |
| Comp. ex. 2 | 87.0 ± 13.0 |

¹⁾compound used in a concentration of 0.01%.
²⁾viability of human corneal epithelial cells measured after treatment with the test compound for 10 minutes in reference to KRB (Krebs-Ringer buffer, 100% viability) and Triton X (0% viability).

The table shows that the compounds of the invention exhibit low toxicity levels and are therefore highly compatible.

### TEER Values (TEER: Transepithelial Electrical Resistance)

TEER is a method to determine the barrier strength of epithelial cells. By determining the barrier strength the effect of a test substance on epithelial cells may be evaluated. TEER values were determined as follows:

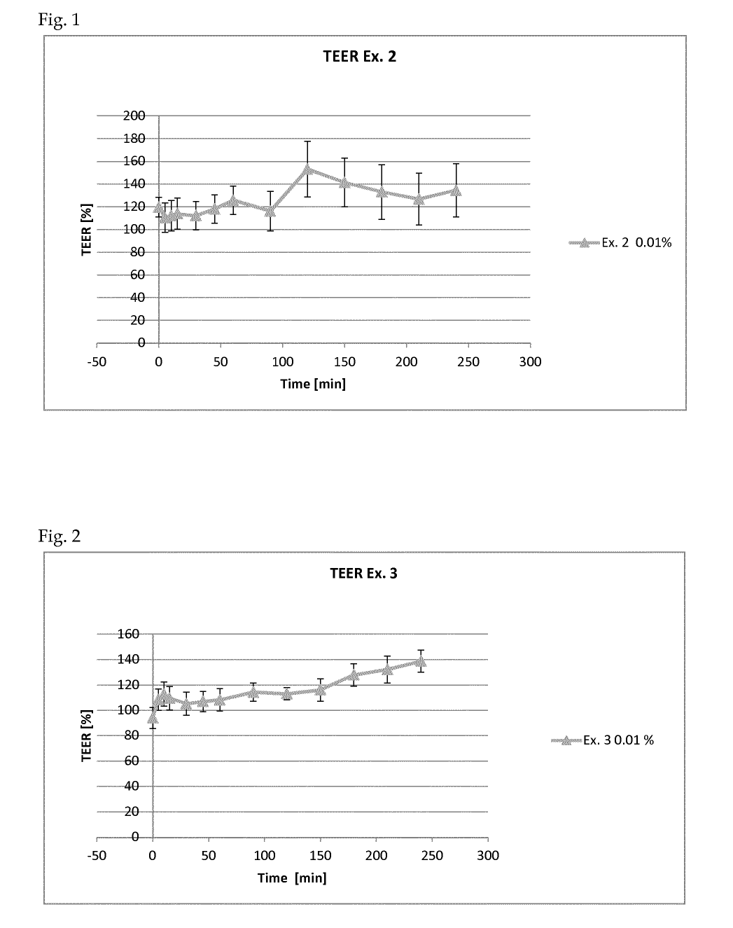

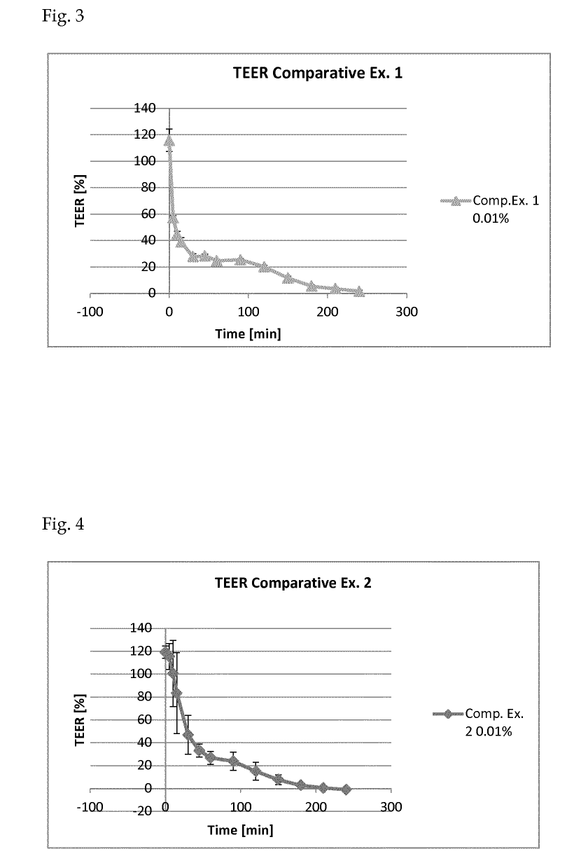

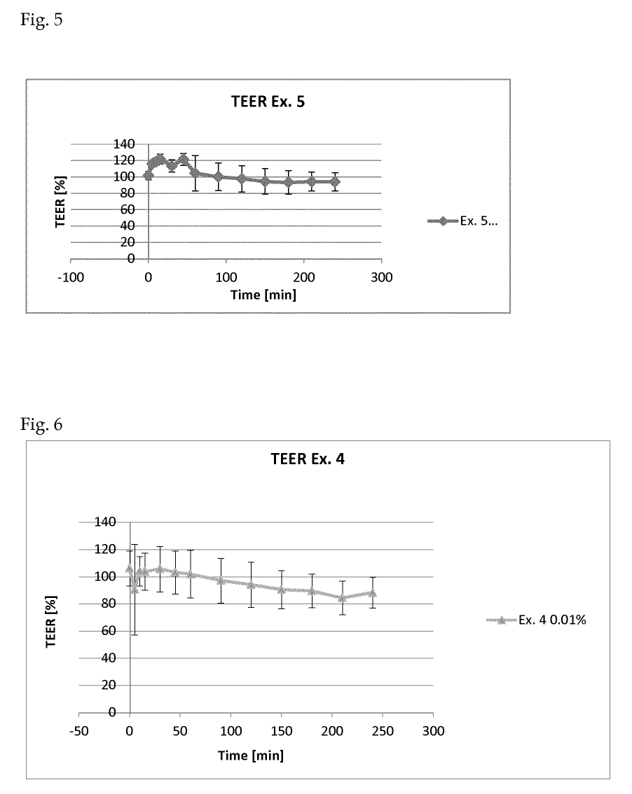

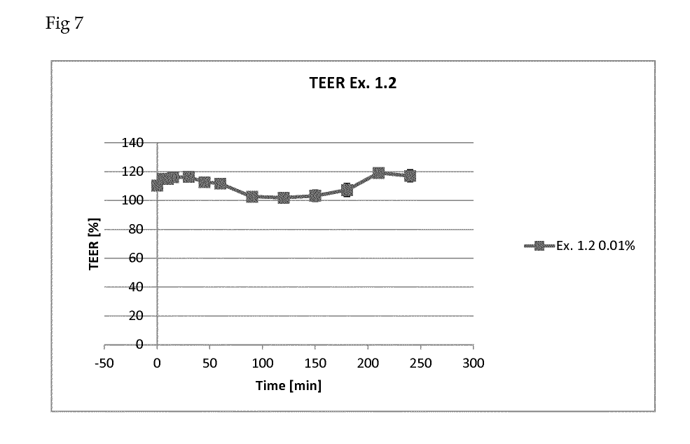

The test was performed with the MDCK-1 cell line using a ThinCert™ cell culture insert in a 24 well format (Greiner Bio-one International GmbH) with 100.000 cells being charged into each well. Underneath the ThinCert™ 1.5 ml and onto the ThinCert™ 0.6 ml cell culture medium were given (MDCK-1 medium containing 500 ml MEM, 10% fetal calf serum, 2 mM L-glutamine and 1% antibiotic). The cells were seeded on day 1 and then incubated at 37° C. and 5% CO₂. On day 3 and 4 the cell culture medium was changed. On day 5 the TEER values were determined using an STX electrode and an Evom measuring device. The initial TEER values were determined (time 0). Thereafter, the cell culture medium was removed from the ThinCerts™ and replaced by prewarmed KRB of pH 7.4 (6.8 g NaCl, 0.4 g KCl, 0.14 g NaH₂PO4×H₂O, 2.1 g NaHCO₃, 3.575 g HEPES, 1.1 D-glucose monohydrate, 0.2 g MgSO₄×7 H₂O, 0.26 g CaCl₂×2 H₂O, aqua bidest. ad 1000 ml). After 30 min the TEER values were again determined. Subsequently, the test substances dissolved in KRB (0.01% and 0.1%) were placed on the upper side of the ThinCerts™. The TEER values were then determined at intervals during 4 hours (5 min, 10 min, 15 min, 30 min, 45 min, 60 min, 1 h 30 min, 2 h, 2 h 30 min, 3 h, 3 h 30 min, 4 h). ThinCerts™ where only KRB was added to the cell layers were used as control. The compounds were tested in a concentration of 0.01%. The results for the compounds of examples 1.2, 2, 3, 4, 5, 6.2, 6.3, and comparative examples 1 to 2 are shown in FIGS. 1 to 7. As can be seen, the TEER values of the compounds of the invention decrease only to a small extent, in particular during the first 90 minutes. This means that the compounds of the invention do not essentially impair the cell barrier so that the cells remain intact and their permeability for impurities etc. is not reduced. This is an essential contribution to the safety of the compounds.

### Further Embodiments of the Invention

1. Cationic compounds of formula (I)

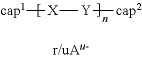

wherein
a) X is

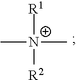

Y is C₂-C₆ alkylene;
cap¹ is

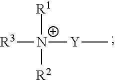

cap² is

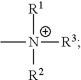

R¹ and R² are independently of each other C₁-C₆ alkyl;
R³ is C₉-C₁₈ alkyl; and
n is 10 to 500, preferably 50-400;
b) X is

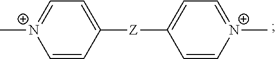

Y is C₄-C₆ alkenylene;
Z is C₁-C₆ alkylene;
cap¹ is

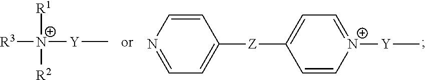

R¹, R² and R³ are independently of each other C₁-C₆ alkyl;
cap² is

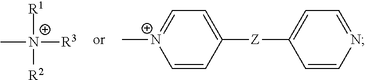

and
n is 1 to 250;
c) X is

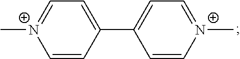

Y is C₂-C₆ alkylene;
cap¹ is

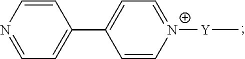

cap² is

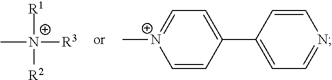

R¹ and R² are independently of each other C₁-C₆ alkyl;
R³ is C₈-C₁₈ alkyl; and
n is 0 to 20; and
d) X is

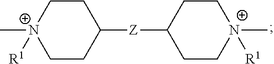

Y is C₂-C₆ alkylene;
Z is C₁-C₆ alkylene which is different from Y;
cap¹ is

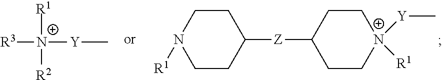

R¹, R² and R³ are independently of each other C₁-C₆ alkyl;
cap² is

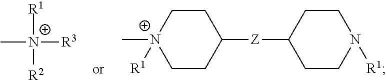

and
n is 5 to 200; and
wherein A is an anion;
u is the valency of the anion; and
r is the number of positive charges in the compound (number of positive charges in group (a): n+2; in group (b): 2n+2; in group (c): 2n+2; in group (d): 2n+2).
2. A cationic compound of embodiment 1, corresponding to the compounds of group (a) having the formula (Ia):

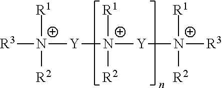

wherein n, Y, R¹, R² and R³ are as defined in embodiment 1.
3. A cationic compound of embodiment 2, wherein R¹ and R² are methyl.
4. A cationic compound of embodiment 2 or 3, wherein R³ is C₁₂-C₁₆ alkyl.
5. A cationic compound of any one of embodiments 2 to 4, wherein Y is C₃-C₄ alkylene.
6. A cationic compound of any one of embodiments 2 to 5, wherein n is 100 to 400, in particular 90 to 140.
7. A cationic compound of embodiment 1, corresponding to the compounds of group (b) having the formula (Ib):

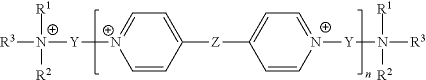

wherein n, Y, Z, R¹, R² and R³ are as defined in embodiment 1.
8. A cationic compound of embodiment 7, wherein R¹, R² and R³ are methyl or ethyl.
9. A cationic compound of embodiment 7 or 8, wherein Y is C₄-alkenylene.
10. A cationic compound of any one of embodiments 7 to 9, wherein Z is C₂-C₄ alkylene.
11. A cationic compound of any one of embodiments 7 to 10, wherein n is 170 to 220.
12. A cationic compound of embodiment 1 corresponding to the compounds of group (c) having the formula (Ic):

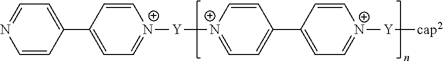

wherein n, Y and cap² are as defined in embodiment 1.
13. A cationic compound of embodiment 12, wherein cap² is

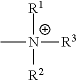

and R¹ and R² are methyl and R³ is as defined in embodiment 1.
14. A cationic compound of embodiment 13, wherein R³ is C₁₀-C₁₆ alkyl.
15. A cationic compound of embodiment 12, wherein cap² is

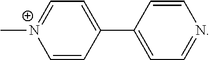

16. A cationic compound of any one of embodiments 12 to 15, wherein Y is C₂-C₄ alkylene.
17. A cationic compound of any one of embodiments 12 to 16, wherein n is 0 to 2.
18. A cationic compound of embodiment 1 corresponding to the compounds of group (d) having the formula (Id):

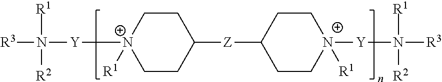

wherein n, Y, R¹, R² and R³ are as defined in embodiment 1.
19. A cationic compound of embodiment 18, wherein R¹, R² and R³ are methyl.
20. A cationic compound of embodiments 18 or 19, wherein Z is C₁-C₃ alkylene.
21. A cationic compound of any one of embodiments 18 to 20, wherein Y is C₄-C₆ alkylene.
22. A cationic compound of any one of embodiments 18 to 21, wherein n is 90 to 140.
23. A composition comprising a cationic compound of any one of embodiments 1 to 22.
24. A composition of embodiment 23 in the form of a disinfectant formulation for medical devices, in particular contact lenses.
25. A composition of embodiment 23 in the form of a preservative formulation for liquid, semi-solid or solid drugs, ophthalmic formulations or cosmetics.
26. An ophthalmic composition comprising a cationic compound of any one of embodiments 1 to 22.
27. A composition of any one of embodiments 23 to 16 which comprises 0.01 to 1.0 wt.-% of a cationic compound of any one of embodiments 1 to 22.
28. A composition of any one of embodiments 23 to 27 which comprises an additional disinfectant or preservative.
29. The use of a cationic compound of any one of embodiments 1 to 22 or of a composition of any one of embodiments 23 to 28 as a disinfectant or preservative.
30. A method of disinfecting or preserving liquid, semi-solid or solid drug formulations, medical devices, in particular contact lenses, or cosmetics which comprises adding a cationic compound of any one of embodiments 1 to 22 or of a composition of any one of embodiments 23 to 28 to said drug formulations or cosmetics or contacting said medical devices, in particular contact lenses, with a cationic compound of any one of embodiments 1 to 22 or of a composition of any one of embodiments 23 to 28.
31. The use of a cationic compound of any one of embodiments 1 to 22 or of a composition of any one of embodiments 23 to 28 as an antimycotic agent.
32. A cationic compound of any one of embodiments 1 to 22 or of a composition of any one of embodiments 23 to 28 for use as an antimycotic agent.
33. The use of a cationic compound of any one of embodiments 1 to 22 or of a composition of any one of embodiments 23 to 28 as an antibacterial agent.
34. A cationic compound of any one of embodiments 1 to 22 or of a composition of any one of embodiments 23 to 28 for use as an antibacterial agent.

1. Cationic compounds of formula (I) 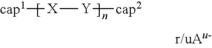
wherein
a) X is 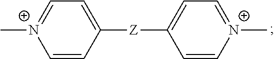
Y is C₄-C₆ alkenylene;
Z is C₁-C₆ alkylene;
cap¹ is 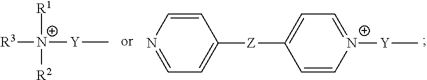
cap is 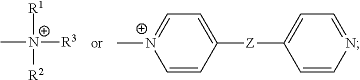
R¹, R² and R³ are independently of each other C₁-C₆ alkyl or C₁-C₆ hydroxyalkyl or one of R¹, R² and R³ is C₈-C₁₈ alkyl and the other two are independently of each other C₁-C₆ alkyl or C₁-C₆ hydroxyalkyl; and
n is 1 to 250;
b) X is 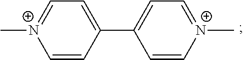
Y is C₂-C₆ alkylene; or
X—Y is a bond
cap¹ is 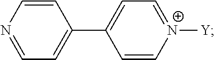
cap² is 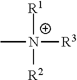
or a halogen atom;
R¹ and R² are independently of each other C₁-C₆ alkyl;
R³ is C₈-C₁₈ alkyl; and
n is 0 to 20; and
c) X is 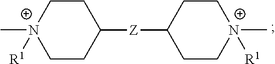
Y is C₂-C₆ alkylene;
Z is C₁-C₆ alkylene which is different from Y;
cap¹ is 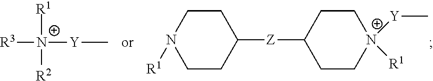
cap² is 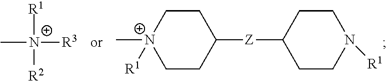
R¹, R² and R³ are independently of each other C₁-C₆ alkyl; and
n is 30 to 150;
d) X is 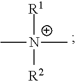
Y is C₂-C₆ alkylene;
cap¹ is 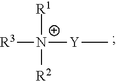
cap² is 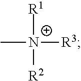
R¹ and R² are independently of each other C₁-C₆ alkyl;
R³ is C₉-C₁₈ alkyl; and
n is 10 to 500;
A is an anion;
u is the valency of the anion; and
r is the number of positive charges in the compound.

2. The cationic compound of claim 1, corresponding to the compounds of group (a) having the formula (Ia): 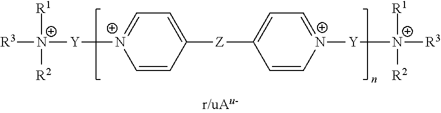
wherein n, r, u, A, Y, Z, R¹, R² and R³ are as defined in claim 1.

3. The cationic compound of claim 2, wherein R¹, R² and R³ are methyl or ethyl, Y is C₄-alkenylene, Z is C₂-C₄ alkylene, and n is 2 to 15.

4. The cationic compound of claim 2, wherein n is 2.

5. The cationic compound of claim 1 corresponding to the compounds of group (b) having the formula (Ib): 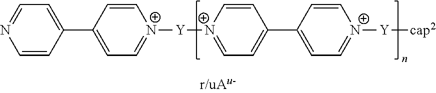
wherein n, r, u, A, Y and cap² are as defined in claim 1.

6. The cationic compound of claim 5, wherein R¹ and R² are methyl, R³ is C₁₀-C₁₆ alkyl, Y is C₂-C₄ alkylene and n is 0 to 10.

7. The cationic compound of claim 6, wherein n is 0.

8. The cationic compound of claim 5, wherein n is 0 and cap² is a halogen atom.

9. The cationic compound of claim 1 corresponding to the compounds of group (c) having the formula (Ic): 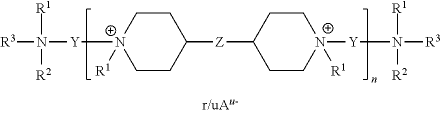
wherein n, r, u, A, Y, R¹, R² and R³ are as defined in claim 1.

10. The cationic compound of claim 9, wherein R¹, R² and R³ are methyl, Z is C₁-C₃ alkylene, Y is C₄-C₆ alkylene and n is 10 to 30.

11. The cationic compound of claim 1, corresponding to the compounds of group (d) having the formula (Id): 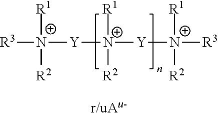
wherein n, r, u, A, Y, R¹, R² and R³ are as defined in claim 1.

12. The cationic compound of claim 11, wherein R¹ and R² are methyl, R³ is C₁₂-C₁₆ alkyl, Y is C₃-C₄ alkylene and n is 50 to 400.

13. The cationic compounds of claim 1 selected from

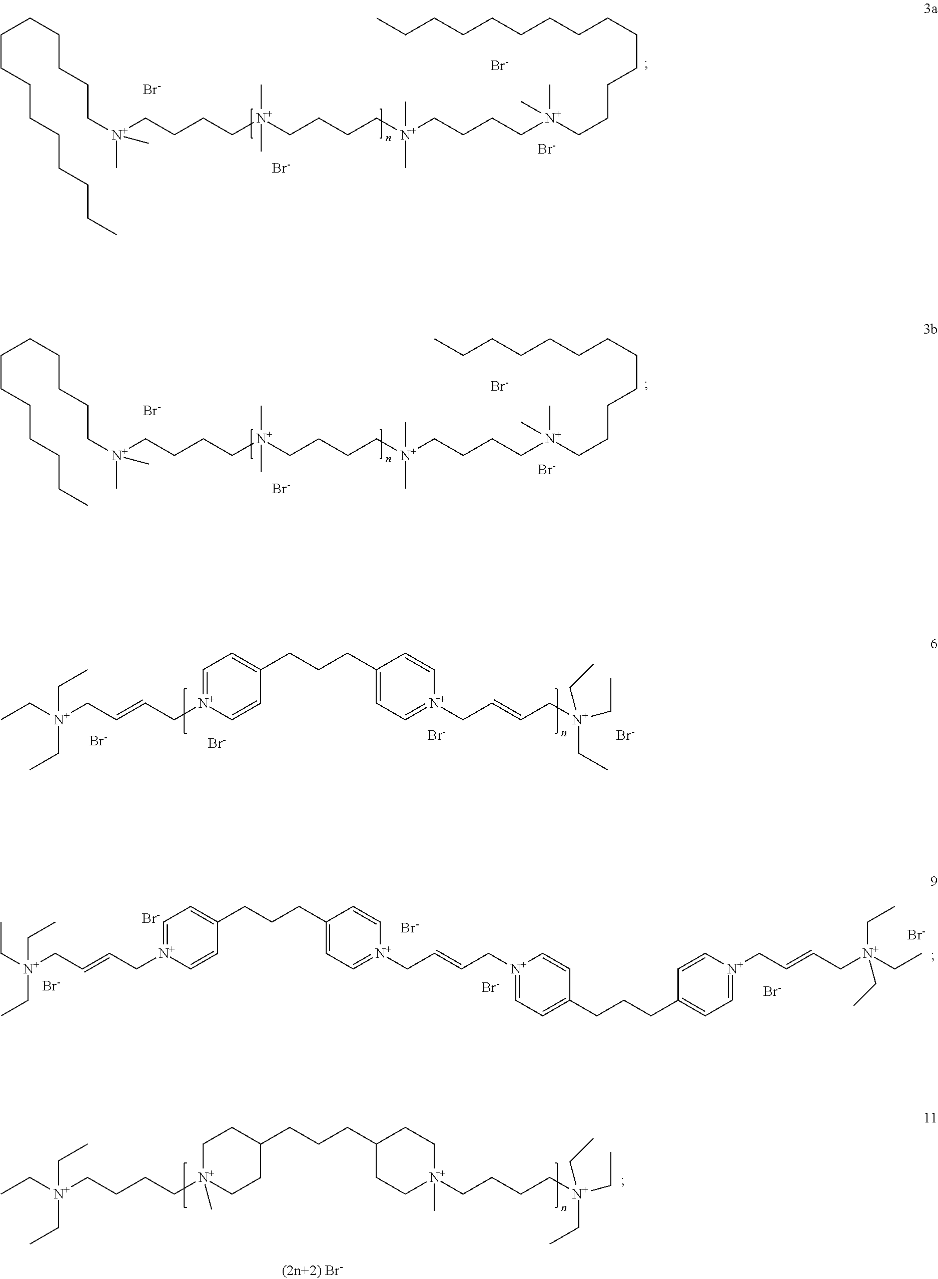

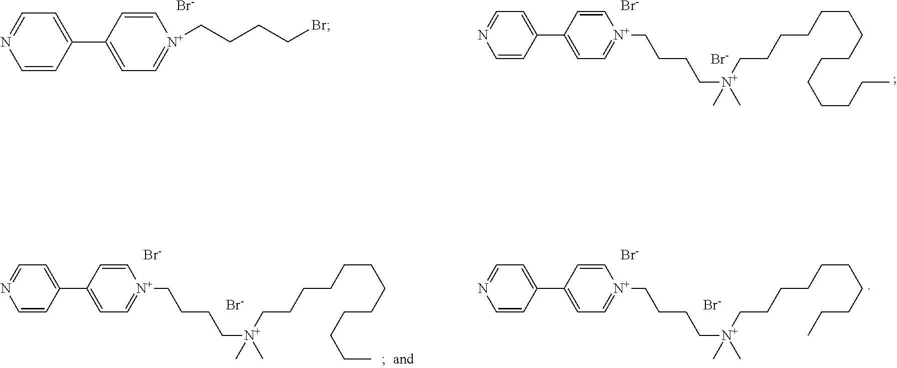

14. A composition comprising a cationic compound of claim 1.

15. The composition of claim 14 in the form of a disinfectant formulation for medical devices or in the form of a preservative formulation for liquid, semi-solid or solid drugs, ophthalmic formulations or cosmetics.

16. An ophthalmic composition comprising a cationic compound of claim 1.

17. The composition of claim 14 which comprises 0.0001 to 1.0 w/v % of the cationic compound.

18. The composition of claim 14 which comprises an additional disinfectant or preservative.

19. A method for disinfecting or preserving comprising the utilization of a cationic compound of claim 1.

20. The method of claim 19 for disinfecting or preserving liquid, semi-solid or solid drug formulations, medical devices, contact lenses, or cosmetics comprising adding a cationic compound of claim 1 to the liquid, semi-solid or solid drug formulation or cosmetic or contacting the medical device or the contact lense, with a cationic compound of claim 1.
